(19) United States
(12) Patent Application Publication
(10) Pub. No.: US 2007/0238870 A1
(43) Pub. Date: Oct. 11, 2007

(54) Hybridization Device and Hybridization Method

(75) Inventors: Mitsuo Kawase, Aichi (JP); Yasuko Yoshida, Aichi (JP); Kazunari Yamada, Aichi (JP); Tomokazu Takase, Gifu (JP)

Correspondence Address: BURR & BROWN, PO BOX 7068, SYRACUSE, NY, 13261-7068, US

(73) Assignee: NGK Insulators, Ltd., Nagoya-City (JP)

(21) Appl. No.: 11/572,506

(22) PCT Filed: Jul. 29, 2005
(86) PCT No.: PCT/JP05/14357

§ 371 Date: Jan. 23, 2007

(30) Foreign Application Priority Data
Jul. 29, 2004 (JP) 2004-221807

(51) Int. Cl. C12M 1/00

(52) U.S. Cl. 536/25.3; 435/285.1

(57) ABSTRACT

The hybridization device of the invention aims to attain a hybridization reaction of high reproducibility. A hybridization device 2 for a hybridization reaction of nucleic acid has a cover member 10 that defines a cavity 12, which includes a nucleic acid fixation area 6 of a substrate 4 for fixation of a nucleic acid probe and has capacity for storage of a liquid for the hybridization reaction therein. At least part of an area exposed to inside of the cavity 12 forms a hydrophobic region 18. Adequate control of the surface characteristic of the area exposed to the cavity for implementing the hybridization reaction desirably enhances the signal intensity and reduces a variation in signal intensity, thus attaining the hybridization reaction of high reproducibility.

### TECHNICAL FIELD

The present invention relates to a hybridization device for a hybridization reaction of nucleic acid, as well as to a corresponding hybridization method and a nucleic acid array.

### BACKGROUND ART

Proposed hybridization devices for a hybridization reaction of nucleic acid on a substrate with a fixed nucleic acid probe, such as a DNA microarray, include the substrate and a flexible layer attached to the substrate as disclosed in Japanese Translation of PCT Application No. 2003-517156 and No. 2003-517591. In these proposed hybridization devices, with a view to reducing an operator-based variation and enhancing the efficiency of the hybridization reaction of the nucleic acid in a small reaction cavity, an external force is applied to a liquid component in the small reaction cavity via the flexible layer by means of a roller to vigorously blend the liquid component.

### DISCLOSURE OF THE INVENTION

The varying signal intensity or the instability of the hybridization reaction mainly arises in the course of the hybridization reaction. Application of the external force to the reaction cavity as the field of the hybridization reaction or to the liquid component included in the reaction cavity undesirably increases the instability of the hybridization reaction, while only insufficiently enhancing the efficiency of the hybridization reaction. No effective measures against such problems have been proposed, and there is still a high demand for eliminating the instability and the unevenness of the hybridization reaction in the small reaction cavity.

An object of the invention is thus to provide a hybridization device to attain a hybridization reaction of high reproducibility and a corresponding hybridization method. An object of the invention is also to provide a hybridization device to attain a hybridization reaction of high accuracy (precision) and a corresponding hybridization method.

As the result of the intensive study and examination, the inventors have found that the signal intensity and its variation (variation coefficient) are significantly affected by the surface characteristic of an area exposed to the reaction cavity for the hybridization reaction and by the structure of the reaction cavity. Adequate control of the surface characteristic and the structure of the reaction cavity has enhanced the signal intensity and reduced its variation to attain the hybridization reaction of high reproducibility. The inventors have completed the invention described below, based on such findings. The present invention is constructed as follows.

A hybridization device for a hybridization reaction of nucleic acid according to one aspect of the invention includes: a cover member that defines a cavity, which includes a nucleic acid probe fixation area of a substrate for fixation of a nucleic acid probe and has capacity for storage of a liquid for the hybridization reaction therein, where at least part of an area exposed to inside of the cavity forms a hydrophobic region. In this hybridization device of the invention, it is preferable that the hydrophobic region is formed in at least part of the cover member. The cover member preferably have the hydrophobic region in an area opposed to the nucleic acid probe fixation area. In this hybridization device of the invention, the hydrophobic region preferably has a water contact angle of not less than 30 degrees.

A hybridization device for a hybridization reaction of nucleic acid according to another aspect of the invention includes: a cover member that defines a cavity, which includes a nucleic acid probe fixation area of a substrate for fixation of a nucleic acid probe and has capacity for storage of a liquid for the hybridization reaction therein, where an opposed area of the cover member facing the nucleic acid probe fixation area has a thickness of not less than 300 μm. In this hybridization device of the invention, the cover member preferably has a hydrophobic region formed in at least part of an area exposed to inside of the cavity.

A hybridization device for a hybridization reaction of nucleic acid according to another aspect of the invention includes: a cover member that defines a cavity, which includes a nucleic acid probe fixation area of a substrate for fixation of a nucleic acid probe and has capacity for storage of a liquid for the hybridization reaction therein, where the nucleic acid probe fixation area included in the cavity has a variation coefficient of spatial height of not higher than 50%. In this hybridization device of the invention, an average spatial height is preferably not less than 15 μm. And, the cover member preferably has a hydrophobic region formed in at least part of an area exposed to inside of the cavity.

In any one of the hybridization device described above, the cover member may have at least a sheet element and a spacer to be interposed between the sheet element and the substrate. In any one of the hybridization device described above, the cover member has an opening for supply of the liquid into the cavity in an area opposed to the nucleic acid probe fixation area, and the opening is preferably configured to have a projected portion formed by outward extension of an inner circumferential wall of the cavity. The cover member preferably has multiple openings located on both ends of the cavity in a longitudinal direction. In any one of the hybridization device described above, an opposed area of the cover member facing the nucleic acid probe fixation area is preferably composed of one or multiple materials selected from the group consisting of polycarbonates, polyolefins, polyamides, polyimides, acrylic resins, fluorides thereof, and poly(vinyl halides).

In any one of the hybridization device described above, an opposed area of the cover member facing the nucleic acid probe fixation area preferably has at least either of a concavity and a convexity. In any one of the hybridization device described above, the cover member may be integrated with the substrate in a detachable manner and may have a tab layer with a holdable, extended end from the substrate and the cover member.

A hybridization method of nucleic acid according to another aspect of the invention includes: a setting step of setting a hybridization device for a hybridization reaction of nucleic acid according to any of claims 1 to 14 to a substrate with a nucleic acid probe fixation area for fixation of a nucleic acid probe; and a hybridization step of implementing a hybridization reaction of an object nucleic acid, which is contained in a liquid supplied to a cavity including the nucleic acid probe fixation area, with the nucleic acid probe in the cavity. In this hybridization method, it is preferable that the hybridization step implements the hybridization reaction while the substrate and the hybridization device stand still. In this hybridization method, the hybridization step preferably moves the specific gas in the cavity to stir the liquid. It is preferable that the hybridization step implements the hybridization reaction while the substrate and the hybridization device defining the cavity is moved. It is also preferable that the hybridization step stirs the liquid in the cavity. Here, the hybridization step implements the hybridization reaction preferably in the presence of a specific gas in the cavity.

A hybridization method of nucleic acid according to another aspect of the invention includes: a hybridization step of implementing a hybridization reaction of an object nucleic acid, which is contained in a liquid supplied to a cavity, with a nucleic acid probe in the cavity, where the cavity includes a nucleic acid probe fixation area of a substrate for fixation of the nucleic acid probe and has capacity for storage of the liquid for the hybridization reaction therein, where the hybridization step moves a specific gas present in the cavity to stir the liquid. It is preferable that the hybridization step implements the hybridization reaction while a member including the substrate and defining the cavity is moved. It is also preferable that the hybridization step implements the hybridization reaction while an external force is applied to the cover member which is made of deformable material and defining the cavity in combination with the substrate.

A hybridization reaction kit for a hybridization reaction of nucleic acid according to still another aspect of the invention includes: a substrate that has a nucleic acid probe fixation area for fixation of a nucleic acid probe; and a cover member that defines a cavity, which includes the nucleic acid probe fixation area of the substrate and has capacity for storage of a liquid for the hybridization reaction therein, where at least part of an area exposed to inside of the cavity forms a hydrophobic region. In this hybridization reaction kit, an opposed area of the cover member facing the nucleic acid probe fixation area preferably has at least either of a concavity and a convexity. The cover member may be integrated with the substrate in a detachable manner and has a tab layer with a holdable, extended end from the substrate and the cover member.

A nucleic acid array according to still another aspect of the invention includes: a substrate that has a nucleic acid probe fixation area with at least one nucleic acid probe fixed therein; and a cover member that defines a cavity, which includes the nucleic acid probe fixation area of the substrate and has capacity for storage of a liquid for a hybridization reaction of nucleic acid therein, where at least part of an area exposed to inside of the cavity forms a hydrophobic region. In this nucleic acid array, the cover member is preferably detachably attached to the substrate. In this nucleic acid array, the cover member may be integrated with the substrate in a detachable manner, and the cover member and the substrate may be laid one upon the other via a tab layer having a holdable, extended end from the substrate and the cover member. An opposed area of the cover member facing the nucleic acid probe fixation area preferably has at least either of a concavity and a convexity.

### BRIEF DESCRIPTION OF THE DRAWINGS

FIG. 1 schematically illustrates the structure of a hybridization device embodying the invention;

FIG. 2 is a plan view and a sectional view of the hybridization device of FIG. 1;

FIG. 3 shows measurement sites for measuring the spatial height;

FIG. 4 shows cDNA spots on a DNA microarray prepared in Example 1;

FIG. 5 is a graph showing the signal intensities of different materials used for the hybridization device;

FIG. 6 is a graph showing the signal intensities of different thicknesses in an opposed area of the hybridization device;

FIG. 7 is a graph showing a plot of variation coefficient of signal intensity against the variation coefficient of spatial height of cavity;

FIG. 8 is a graph showing a plot of variation coefficient of signal intensity against the spatial height of cavity;

FIG. 9 illustrates a cover member with a tab layer and an array;

FIG. 10 is a graph showing a change of the signal intensity by stirring; and

FIG. 11 shows a cover member and an array in Example 6.

### BEST MODES OF CARRYING OUT THE INVENTION

The hybridization device of the invention is applied to a hybridization reaction of nucleic acid. The hybridization device has a cover member that defines a cavity, which includes a nucleic acid probe fixation area of a substrate for fixation of a nucleic acid probe and has capacity for storage of a liquid for the hybridization reaction therein. In a first embodiment of the hybridization device, at least part of an area exposed to inside of the cavity forms a hydrophobic region. The hybridization device of the first embodiment desirably accelerates the hybridization reaction and enhances the detected intensity (signal intensity) of the resulting hybridized product. This enables the efficient hybridization reaction over the whole nucleic acid probe fixation area and thus enhances the reproducibility of the hybridization reaction. The acceleration of the hybridization reaction in the presence of the hydrophobic region in the small cavity for storage of the liquid for the hybridization reaction of the nucleic acid was far better than the expectation of the inventors. Although the principle of the invention is not theoretically restrained, this better-than-expected effect may be ascribed to the deduction that the presence of the hydrophobic region formed in the cavity accelerates convection of the liquid and diffusion of the object nucleic acid contained in the liquid and accordingly enhances the probability of contact and the hybridization reaction of the object nucleic acid with the nucleic acid probe.

In a second embodiment of the hybridization device, an opposed area of the cover member facing the nucleic acid probe fixation area has a thickness of not less than 300 μm. The hybridization device of the second embodiment desirably reduces a variation in signal intensity of the resulting hybridized product and accordingly enhances the detection accuracy and the reproducibility of the hybridization reaction. Although the principle of the invention is not theoretically restrained, these effects maybe ascribed to the deduction that the opposed area of the cover member facing the nucleic acid probe fixation area having the thickness of not less than 300 μm has a certain heat capacity to work a thermal buffer against the heated liquid stored in the cavity and accordingly enables the hybridization reaction to proceed substantially homogeneously at multiple different locations on the substrate.

In a third embodiment of the hybridization device, the nucleic acid probe fixation area included in the cavity has a variation coefficient of spatial height of not higher than 50%. The hybridization device of the third embodiment desirably reduces a variation in signal intensity of the resulting hybridized product and accordingly enhances the detection accuracy of the hybridization reaction. Although the principle of the invention is not theoretically restrained, these effects may be ascribed to the deduction that the nucleic acid probe fixation area in the cavity having the variation coefficient of spatial height of not higher than 50% reduces the potential adverse effect of the surface characteristic and the morphology of the internal surface of the cavity on the convection of the liquid and on the diffusion of the object nucleic acid contained in the liquid and accordingly enables the hybridization reaction to proceed substantially homogeneously at multiple different locations on the substrate.

The hybridization method of the invention includes a hybridization step of implementing a hybridization reaction of an object nucleic acid, which is contained in a liquid supplied to a cavity, with a nucleic acid probe in the cavity. The cavity includes a nucleic acid probe fixation area of a substrate for fixation of the nucleic acid probe and has capacity for storage of the liquid for the hybridization reaction therein. The hybridization step moves a specific gas present in the cavity to stir the liquid. The hybridization method of the invention desirably increases the efficiency of the hybridization reaction, thus enhancing the signal intensity of the resulting hybridized product and reducing a variation in signal intensity.

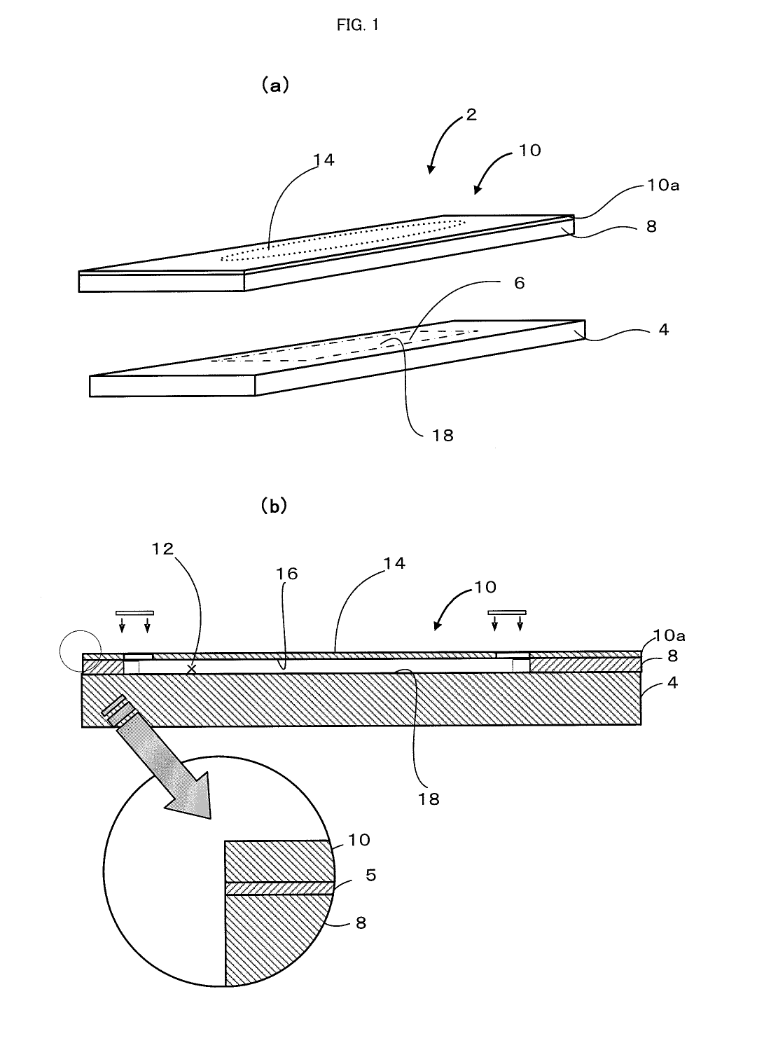

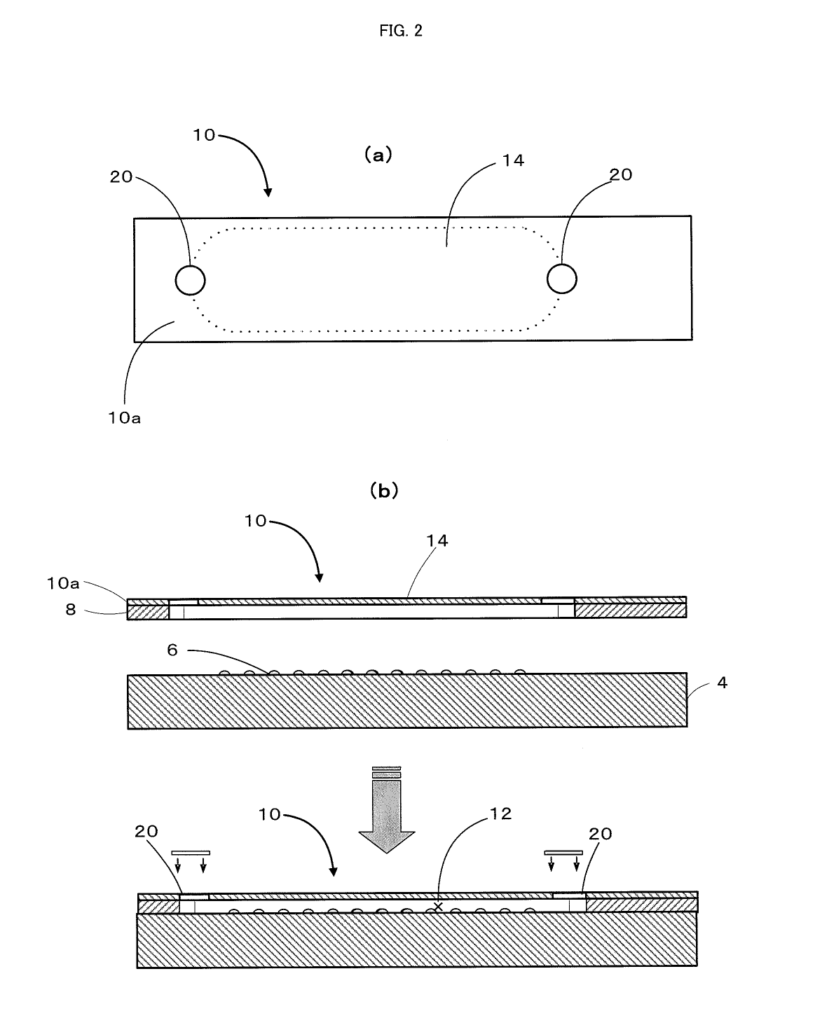

The present invention will be better understood by the following detailed description of the hybridization devices of the first through the third embodiments, the hybridization methods using these hybridization devices, and corresponding hybridization kits with reference to the accompanied drawings. FIG. 1 illustrates a typical structure of the hybridization device of the invention with a substrate. FIG. 2 is a plan view and a sectional view of the hybridization device.

Hybridization Device

A hybridization device 2 is applied to a hybridization reaction of nucleic acid. The nucleic acid is to be at least partly hybridized with another nucleic acid by base-pairing. The terminology ‘nucleic acid’ thus conceptually includes natural and synthetic nucleotide oligomers and nucleotide polymers, DNAs including genome DNAs and cDNAs, PCR products, RNAs including mRNAs, and peptide nucleic acids. The hybridization reaction means binding of complementary strands of nucleic acid molecules by base-pairing.

Substrate

A substrate 4 processed by the hybridization device 2 has a nucleic acid fixation area 6 for fixation of at least one nucleic acid probe. The nucleic acid fixation area 6 is provided or prepared for formation of one or multiple small regions (spots) respectively with fixed nucleic acid probes. The present invention does not specifically define the method or the conditions for fixation of the nucleic acid probe to the substrate 4, but any of the known techniques and conditions at the time of this application maybe adopted for this purpose. The nucleic acid fixation area 6 of the substrate 4 may include small three-dimensional spots but is preferably configured to be practically flat. One or multiple nucleic acid fixation areas 6 may be provided directly on the substrate 4 or may be placed on the substrate 4 via a medium, for example, a porous medium, according to the requirements. The multiple nucleic acid fixation areas 6 formed on the substrate 4 may be mutually separated via hydrophobic partitions.

The substrate 4 may have any shape; for example, a flat plate or a flat bottom of a concave body may function as the substrate 4. The substrate 4 may be made of any of conventionally used and other materials. Available materials are silicon- and other ceramics including glass, silicon dioxide, and silicon nitride, resins including silicone, polymethyl methacrylate, and poly(meth)acrylates, and metals including gold, silver, and copper. The selected material may be covered with an appropriate coating agent to have desired surface characteristics. Glass substrates, silicone substrates, and acrylic resin substrates are commonly used for the substrate 4. The most typical example of the substrate 4 is a substrate for a DNA chip or a DNA microarray having fixed cDNA probes or to have fixed cDNA probes.

Cover Member

The hybridization device 2 has a cover member 10 to cover over the substrate 4. The cover member 10 defines a cavity 12 for the hybridization reaction, which includes the nucleic acid fixation area 6 of the substrate 4. The cover member 10 may be attached to the substrate 4 or may be attached to a substrate holder for receiving or holding the substrate 4 therein, in order to define the cavity 12 by combination with the substrate 4. In the former case, the cover member 10 is attached to a flat plate substrate or to a flat bottom of a concave body substrate. In the latter case, the cover member 10 is attached to a substrate holder having a flat portion or a concave portion for placing a flat plate substrate therein.

The cavity 12 is the space including the nucleic acid fixation area 6 and has capacity for storage of a liquid for the hybridization reaction (hereafter referred to as the hybridization liquid). The cavity 12 preferably has a space of a preset spatial height (spatial thickness) above the nucleic acid fixation area 6. Namely the cover member 10 is configured to have an opposed area 14 facing the nucleic acid fixation area 6 of the substrate 4 across a certain distance even in the absence of the hybridization liquid. The cover member 10 of such configuration enables formation of the cavity 12 having the preset spatial height over the nucleic acid fixation area 6 by simple attachment of the cover member 10 to the substrate 4 or the substrate holder without any special operations.

At least the nucleic acid fixation area 6 of the substrate 4 and the opposed area 14 of the cover member 10 are exposed to the cavity 12. There is an additional face exposed to the cavity 12 to confine the cavity 12 from the outside. The additional face may be part of the substrate 4 or part of the cover member 10 or may be a separate element. The shape of the cavity 12 is not specifically restricted but desirably has no projection or corner that may cause retention of the hybridization liquid. An external projection or extension may, however, be allowed as long as it is sufficiently small and is formed in the vicinity of a curved side wall that prevents retention of the liquid. The desirable shape of the cavity 12 is an ellipse or a circle as shown in FIGS. 1 and 2. The opposed area 14 of the cover member 10 exposed to the cavity 12 may be convex or concave but is preferably flat. The flat opposed area 14 facilitates formation of the cavity 12 having substantially the fixed spatial height over the nucleic acid fixation area 6.

In the illustrated example of FIG. 1, the substrate 4 is a flat plate, and the cover member 10 has a spacer 8 of a predetermined height formed around the periphery of a flat plate element 10a including the opposed area 14. Namely the cover member 10 of FIG. 1 includes the flat plate element 10a having practically the same surface dimensions as those of the substrate 4 and a substantially flat plane on at least one side facing the substrate 4, and the spacer 8 formed around the periphery of the flat plate element 10a to be interposed between the substrate 4 and the flat plate element 10a. Otherwise the cover member 10 may have a predetermined dome shape to cover over the nucleic acid fixation area 6. The cover member 10 may be a molded body of polymer material. In the structure of the nucleic acid fixation area 6 placed in the bottom of a recess, the cover member 10 may be a flat plate attached to the upright circumferential wall of the recess. Some examples of this structure are the substrate 4 of a concave body having the nucleic acid fixation area 6 formed on the bottom of a recess, the substrate 4 having a peripheral side wall of a predetermined height around the nucleic acid fixation area 6, and the substrate 4 placed in the bottom of a substrate holder.

Available materials of the spacer 8 include acrylic resins, thermoplastic elastomers, natural and synthetic rubbers, silicone, polyolefins, polyamides, polyimides, vinyl halides, and polycarbonates.

Hydrophobic Region

The hybridization device 2 has a hydrophobic region 16 in at least part of the area exposed to the cavity 12. The terminology ‘hydrophobic’ here represents a surface characteristic having at least the water repellency. The hydrophobic region 16 preferably has the higher water repellency than general sodium silicate glass without any hydrophilic treatment. The water repellency is generally expressed by a water contact angle on the flat surface. The water contact angle of the hydrophobic region in the present invention is not less than 30 degrees, preferably not less than 60 degrees, more preferably not less than 70 degrees, or most preferably not less than 90 degrees. The water contact angle represents a contact angle of a droplet placed on a level solid plane. The contact angle may be a static contact angle, an advanced or backward contact angle as the critical value, or a dynamic contact angle, but is preferably a static contact angle measured by the drop method.

There are three conventional techniques adopted for the drop method of measuring the static contact angle: (1) tangent method, (2) θ/2 method, (3) three-point click method. The tangent method (1) adjusts the cursor of a reading microscope on the tangent of a droplet to directly measure the contact angle. The θ/2 method (2) doubles the angle of a line between one end and an apex of a droplet to the solid surface to specify the contact angle. The three-point click method (3) clicks an apex of a droplet and two contact points of the droplet with the solid surface on a computer image and specifies the contact angle by image processing. Among these three methods, the method (2) or the method (3) is preferably applied to determine the contact angle in the present invention.

The hydrophobic region 16 is formed in at least part of the area exposed to the cavity 12 and is preferably provided on the cover member 10. The hydrophobic region 16 formed on the cover member 10 effectively enhances the signal intensity. The hydrophobic region 16 is preferably formed in the opposed area 14 of the cover member 10. More specifically the uniform hydrophobic region 16 is formed over the whole opposed area 14 corresponding to substantially the whole nucleic acid fixation area 6. Although the opposed area 14 may have multiple discrete hydrophobic regions 16, it is preferable to form one continuous hydrophobic region 16 over the substantially whole opposed area 14. The whole exposed area of the cover member 10 exposed to the cavity 12 may form the hydrophobic region 16.

The cover member 10 may be made of a hydrophobic material to form the hydrophobic region 16. Alternatively only a specific area of the cover member 10 corresponding to the hydrophobic region 16 may be composed of the hydrophobic material. Otherwise the specific area of the cover member 10 may be subjected to certain surface treatment to give the hydrophobic characteristic (water repellency). Available examples of the hydrophobic material for the hydrophobic region 16 are polycarbonates, polyolefins including polyethylene and polypropylene, vinyl halides, polyamides, polyimides, acrylic resins, and fluorides and chlorides of these resins or polymers. The surface treatment to give the water repellency is, for example, chemical modification or mechanical processing of a certain material surface to be roughened and have the contact angle of not less than 90 degrees.

The distance between the nucleic acid fixation area 6 and the opposed area 14 in the cavity 12, that is, the spatial height over the nucleic acid fixation area 6 in the cavity 12 preferably has a variation coefficient (standard deviation/average×100 (%)) of not higher than 50%. The variation coefficient of spatial height of not higher than 50% at the nucleic acid fixation area 6 desirably reduces a variation in signal intensity of the resulting hybridized product. The reduced variation of the signal intensity enables detection of high accuracy and attains the hybridization reaction of high reproducibility. The variation coefficient of spatial height of not higher than 50% readily lowers the variation coefficient of signal intensity to or below 20%. The variation coefficient of spatial height is preferably not higher than 40%, more preferably not higher than 30%, and most preferably not higher than 20%. According to the inventors' findings, the variation coefficient of spatial height of or below a predetermined level at the nucleic acid fixation area 6 in the cavity 12 has significant contribution to the evenness of the amount of the hybridization liquid (liquid thickness) per unit area of the nucleic acid fixation area 6.

The spatial height of the cavity 12 preferably has an average of not less than 15 μm. The average spatial height of not less than 15 μm desirably reduces a variation in signal intensity of the resulting hybridized product. More specifically the average spatial height is not less than 20 μm. The spatial height of not less than 20 μm in the cavity 12 allows a certain liquid thickness on the nucleic acid fixation area 6 and thus ensures convection of the hybridization liquid and diffusion of an object nucleic acid contained in the hybridization liquid, while controlling the potential effects of the exposed area to the cavity 12. The average spatial height preferably has an upper limit of 1000 μm. The parted area of the substrate 4 corresponding to the cavity 12 is preferably in a range of 1 mm² to 2000 mm².

The average and the variation coefficient of the spatial height in the cavity may be determined by height-surface undulation measurement method described below.

(1) Measurement Sites

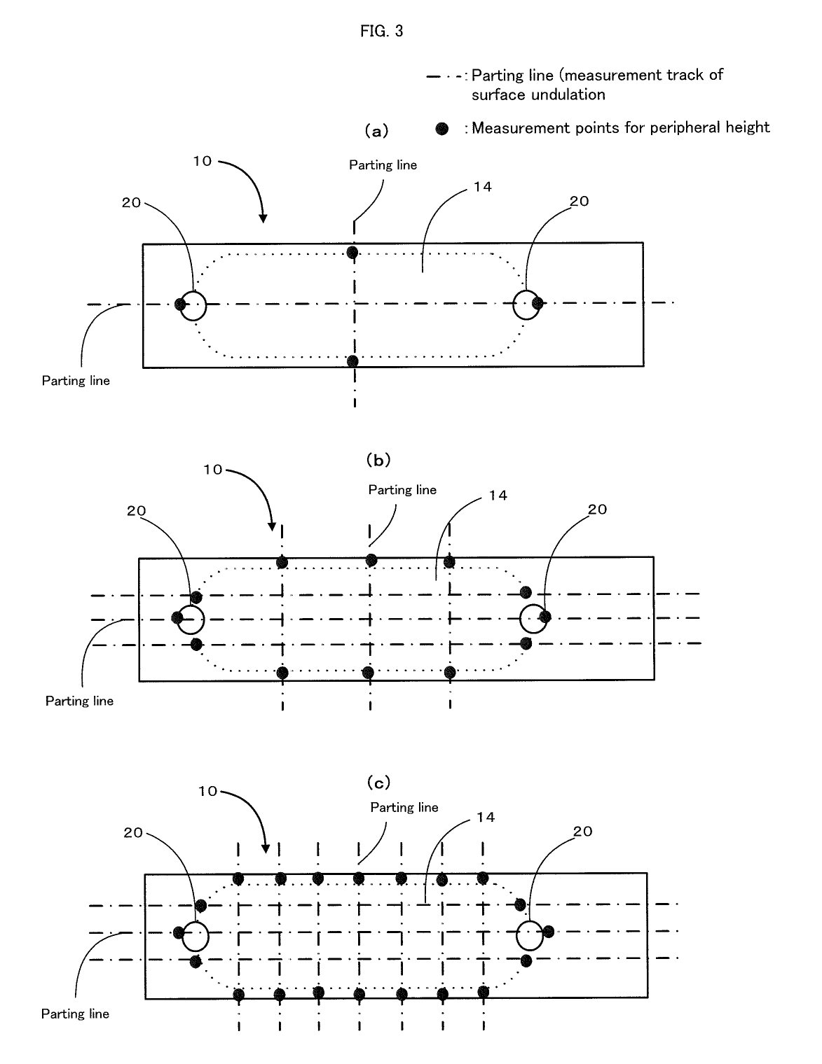

The measurement sites are on parting lines for dividing the cavity 12 defined by the cover member 10 or more specifically on the center line and on equally-divided parting lines of the cavity 12. In one example shown in FIG. 3(a), the measurement sites are 2 parting lines that divide the cavity 12 into two equal parts both in the lateral direction and in the vertical direction. In another example shown in FIG. 3(b), the measurement sites are 6 parting lines that divide the cavity 12 into four equal parts both in the lateral direction and in the vertical direction. Instill another example shown in FIG. 3(c), the measurement sites are 10 parting lines that divide the cavity 12 into eight equal parts in the vertical direction and into four equal parts in the lateral direction.

(2) Measurement of Peripheral Height and Computation of Reference Height

A reference height (H) is determined first. The reference height (H) represents an average of height (peripheral height) from the surface of the substrate 4 including the nucleic acid fixation area 6 to the periphery of the cover member 10 corresponding to the circumferential part of the cavity 12 formed by attachment of the cover member 10 to face the nucleic acid fixation area 6 of the substrate 4. The peripheral height is measured at peripheral points on each parting line as shown in FIG. 3. Each parting line divides the cavity 12, so that the peripheral height is measured at two opposed peripheral points on each parting line. Namely the total number of measurement points for the peripheral height is equal to the number of parting lines×2. The average of measurements of the peripheral height on all the parting lines is calculated and is defined as the reference height (H) For calculation of the average spatial height and its variation coefficient, the number of measurement points for the peripheral height is preferably not less than 4 or more specifically not less than 20.

(3) Measurement of Surface Undulation of Cover Member

The surface undulation of the cover member 10 is measured as a variation in surface convex and concave on each parting line relative to the circumference of a certain area on an outer surface of the cover member 10 (that is, a surface that does not face the substrate 4) corresponding to the opposed area 14. The maximum and the minimum of the surface undulation are measured on each parting line as the measurement traction. Namely there are two measurement points for the surface undulation on each parting line. The total number of measurement points for the surface undulation is equal to the number of parting lines×2. For calculation of the average spatial height and its variation coefficient, the number of measurement points for the surface undulation is preferably not less than 4 or more specifically not less than 20.

(4) Measurement of Film Thickness

The film thickness of the cover member 10 represents the film thickness of the opposed area 14. The film thickness used here may be an average film thickness (Tave) of the opposed area 14 or may be film thicknesses (Tmax, Tmin) at measurement points of the maximum and the minimum of surface undulation, although the use of the average film thickness (Tave) is preferable. The film thickness is measured with any known measurement instrument, for example, a slide caliper.

(5) Computation of Spatial Height

Multiple spatial heights on each parting line are computable from these measured data. Maximum and minimum spatial heights on each parting line are determinable from the maximum and minimum surface undulations (MAX and MIN):

Maximum Spatial Height=Reference Height (H)+Maximum Surface Undulation (MAX)−Film Thickness (Tave or Tmax)

Minimum Spatial Height=Reference Height (H)+Minimum Surface Undulation (MIN)−Film Thickness (Tave or Tmin)

The maximum spatial height and the minimum spatial height obtained for all the parting lines are averaged to give an average spatial height, and standard deviation/average spatial height×100 gives a variation coefficient (%)

The peripheral heights of the cover member 10 at predetermined positions may be measured with a digital micrometer (Digimicro manufactured by Nikon Corporation), and the surface undulations of the cover member 10 may be measured with a surface texture and contour measuring instrument (Surfcom manufactured by Tokyo Seimitsu Co., Ltd).

The volume of the cavity 12 is adequately designed according to the requirements but is preferably in a range of 0.1 μl to 2000 μl and is more preferably in a range of 1 μl to 1000 μl.

A specific portion of the cover member 10 including the opposed area 14 preferably has the optical transparency to enable the external observation of the inside of the cavity 12. The specific portion including the opposed area 14 preferably has an average thickness of not less than 300 μm. The average thickness of not less than 300 μm well controls a variation coefficient of signal intensity of the hybridized product. The average thickness of not less than 350 μm is more preferable. The upper limit of the average thickness is not specifically defined but is preferably not greater than 3000 μm, since the excessive thickness leads to an excess heat capacity and may cause an uneven temperature distribution in the cavity under heating.

Other Structural Feature

The cover member 10 has openings 20 for injection of the hybridization liquid. There are preferably two or more openings 20, and at least one of the openings 20 is open in the vicinity of the contour for defining the cavity 12 in the cover member 10. This location of the opening 20 prevents the hybridization liquid injected into the cavity 12 from retaining on the inner wall of the cavity 12 but facilitates diffusion of the hybridization liquid over the whole cavity 12. The openings 20 are preferably formed along the contour of the cavity 12 and are more specifically formed as outward extensions from the inner wall of the cavity 12. In the illustrated structure of FIG. 1, two circular openings 20 in the cover member 10 are located at both ends of the elliptic cavity 12 in the longitudinal direction and are formed to be partially projected as extensions from the respective end walls of the cavity 12. This design of the openings 20 ensures sufficient diffusion of the hybridization liquid injected through the openings 20 in the cavity 12. The openings 20 are sealed with adequate sealing members.

The cover member 10 used for the hybridization device 2 is manufactured by combining the spacer 8 with the flat plate element 10a as the main body of the cover member 10 via a sealing layer 5. The sealing layer 5 may be an adhesive or binding layer for bonding the flat plate element 10a to the spacer 8. Attachment of multiple spacers 8 for parting adjacent space divisions to one cover member 10 readily defines multiple cavities 12 between the substrate 4 and the cover member 10.

The cover member 10 may not be an assembled body but may be an integrally molded resin body. The cover member 10 preferably has an adhesive or binding layer at a specific site for attachment to the substrate 4 or to the substrate holder. The adhesive layer is desirably protected by a detachable sheet. Another application of the invention is a hybridization reaction kit including the cover member 10 and the substrate 4. Fixation of a nucleic acid probe to the substrate 4 of this hybridization reaction kit gives an effective nucleic acid array. The cover member 10 may be provided separately from the substrate 4 or the substrate holder. The cover member 10 may be bonded to the substrate 4 or the substrate holder or may be molded as an integral body with the substrate 4 or the substrate holder. The cover member 10 may be detachably attached to the substrate 4 or the substrate holder for the convenience of cleaning and signal detection.

The specific portion of the cover member 10 including the opposed area 14 may have elastic deformability. The specific portion of the cover member 10 or the whole cover member 10 may be made of an elastically deformable material. Application of a gas pressure or mechanical external force to the opposed area 14 elastically deforms the specific portion or the whole cover member 10 to stir the hybridization liquid in the cavity 12.

An exposed side of the opposed area 14 of the cover member 10 exposed to the cavity 12 (that is, a side facing the substrate 4) may have concaves and/or convexes. These concaves and/or convexes give the complicated flow of the hybridization liquid and raise the stirring efficiency of the hybridization liquid in the cavity 12, thus enhancing the hybridization efficiency. The concaves and/or convexes may be formed integrally with the opposed area 14 of the cover member 10 or may be obtained by application of a film or sheet with undulated surface on the side of the cover member 10 facing the substrate 4. The concaves and/or convexes may be any dimensions set according to the spatial height of the cavity 12. The concaves and/or convexes may have a hydrophobic area.

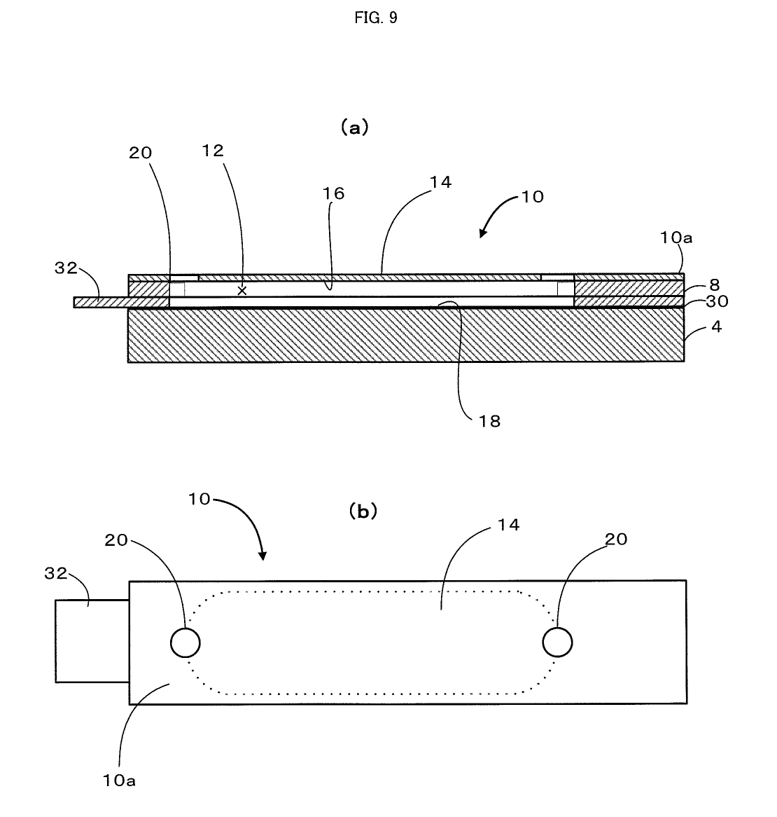

In another structure shown in FIG. 9, the cover member 10 is laid on and integrated with the surface of the substrate 4 including the nucleic acid fixation area 6. A tab layer 30 is interposed between the cover member 10 and the substrate 4 to allow detachment of the cover member 10 from the substrate 4. The tab layer 30 preferably has an extended end 32 from the edges of the substrate 4 and the cover member 10 assembled for defining the cavity 12. The extended end 32 has a length of extension suitable for holding. The tab layer 30 is provided to be at least detachable from the substrate 4. A face of the tab layer 30 on the side of the substrate 4 has a certain level of adhesiveness to allow later detachment from the substrate 4 or is attached to the substrate 4 via an adhesive layer having the certain level of adhesiveness. For detachment of the cover member 10 from the substrate 4, for example, after completion of the hybridization reaction, the extended end 32 of the tab layer 30 is held and pulled outward. This destroys the attachment of the tab layer 30 to the substrate 4 and accordingly detaches the cover member 10 from the substrate 4. This arrangement ensures easy removal of the cover member 10 without application of significant loading onto the substrate 4.

The extended end 32 of the tab layer 30 may be held with fingers or with an adequate tool. The extended end 32 may be formed at only one part of the tab layer 30 or may be formed around the periphery of the assembly of the substrate 4 and the cover member 10. The tab layer 30 is preferably made of a material having the certain level of adhesiveness to allow later detachment from the substrate 4, for example, a resin material or a silicone or another rubber material. This arrangement does not require any separate adhesive layer formed between the tab layer 30 and the substrate 4. The tab layer 30 may be or may not be part of the cover member 10. The tab layer 30 may be provided to be additionally detachable from the cover member 10. This structure further relieves the potential loading applied on the substrate 4.

Hybridization Method of Nucleic Acid

The hybridization reaction is performed according to the conventional procedure with the hybridization device 2 described above. The hybridization process with the hybridization device 2 first attaches the cover member 10 with a sealing layer on the side facing the substrate 4 to a DNA microarray as the substrate 4 via the sealing layer, injects a hybridization liquid prepared by a preset method through the two openings 2, seals the two opening 20 with the sealing members, and causes the DNA microarray with the cover member 10 to stand still at temperature of not lower than 25° C. and not higher than 80° C. for a preset time period.

The hybridization device 2 has the hydrophobic region 16 in at least part of the exposed area to the cavity 12. The presence of the hydrophobic region 16 promotes convection of the hybridization liquid and diffusion of the object nucleic acid contained in the hybridization liquid in the cavity 12 without application of any external force to the substrate 4, for example, stirring, vibration, abrasion, or jet flow, to accelerate the hybridization reaction and enhance the efficiency of the hybridization reaction. The hybridization device 2 including the cover member 10 is preferably applied to the hybridization method that performs the hybridization reaction in the stand-still condition, as well as to various test methods including the hybridization process. The stand-still condition has the sufficient effect of accelerating the hybridization reaction. The hybridization device 2 thus ensures the hybridization result of high reproducibility, while reducing or even eliminating operator-based variations caused by the different handling operations of the substrate 4 and the cover member 10 and external environment-based variations caused by, for example, the levelness of the hybridization device 2 for the stand-still hybridization reaction and the magnitude of the external force.

Controlling the spatial height and its variation coefficient of the cavity 12 defined by the substrate 4 and the cover member 10 of the hybridization device 2 and the thickness of the opposed area 14 of the cover member 10 reduces the variation in signal intensity of the hybridized product and enables highly accurate signal detection. The simple control of the structure and the dimensions of the cavity 12 attains the effect of reducing the variation in signal intensity of the hybridized product without any complicated technique conventionally adopted for the same purpose. The control of the structure and the dimensions of the cavity 12 also has the thermal buffer effect and the effect of substantially equalizing the amount of the hybridization liquid per unit area of the nucleic acid fixation area 6.

The presence of the concaves and/or convexes on the side of the opposed area 14 of the cover member 10 facing the substrate 4 ensures the desired convection of the hybridization liquid in the stand-still condition and enhances the efficiency of the hybridization reaction.

The hybridization method may have a stirring step to stir the hybridization liquid in the cavity 12 defined by the substrate 4 and the cover member 10. The hydrophobic region 16 is preset at least in part of the cavity 12. The aqueous liquid stirred in the cavity 12 is repelled by the hydrophobic region 16. This accelerates the movement of the hybridization liquid in the cavity 12 and further enhances the efficiency of the hybridization reaction.

The movement of the hybridization device 2 including the substrate 4 is effective for stirring the hybridization liquid in the cavity 12. For example, the substrate 4 and the relevant members for defining the cavity 12 may be rotated, swirled, seesawed, reciprocated, turned upside down, or moved by combination of any two or more of such actions. When the opposed area 14 of the cover member 10 is made of the elastically deformable material, deformation of the opposed area 14 by an external force stirs the hybridization liquid in the cavity 12. In one example, a roller or another rotating member may be moved with rotation on the opposed area 14. In another example, a pressing member may be moved with application of pressure on the opposed area 14. Such active stirring may be continued throughout the hybridization process or may be performed intermittently or only in part of the hybridization process.

For effectively stirring the hybridization liquid in the cavity 12, the cavity 12 includes a gas insoluble in the hybridization liquid (for example, the air or an inert gas like nitrogen), in addition to the hybridization liquid. While the cavity 12 stands still, the gas in the cavity 12 is generally retained in a fixed position. The hybridization reaction does not vigorously proceed in this gas retention area (gas accumulation). Application of an external force to move the hybridization liquid in the cavity 12 in the presence of the gas desirably promotes the movement of the hybridization liquid in the cavity 12 and thus accelerates the hybridization reaction.

The hydrophobic region 16 present in at least part of the cavity 12 repels the aqueous liquid and promotes the movement of the gas accumulation to enhance the stirring effect. The presence of the concaves and/or convexes on the side of the opposed area 14 of the cover member 10 facing the substrate 4 changes the moving range of the gas accumulation in the cavity 12, thus further enhancing the stirring effect.

In the presence of the gas in the cavity 12, it is desirable to move the substrate 4 by means of one or multiple gas accumulations in the cavity 12 as the stirrer. The gas accumulation shifts in the cavity 12 to move the substrate 4, while substantially keeping its shape. This ensures effective acceleration of the hybridization reaction. The state of ‘substantially keeping the shape of the gas accumulation’ means that the gas is moved or retained in the cavity 12 in the shape of one or multiple gas accumulations in the major portion of the stirring process. The vigorous shaking of the substrate 4 to disperse the gas over the hybridization in the cavity 12 can not be predominant in the stirring process. The gas accumulation may be temporarily split and gathered again or may be temporarily dispersed over the hybridization liquid in the course of movement in the cavity 12.

The preferable movement of the gas accumulation in the cavity with substantially keeping its shape is the rotational motion, the seesaw motion, or its combination. Such motions enable stable movement of the gas accumulation in the cavity 12. For example, the rotational motion may be made by a slewing mechanism including a vertical rotor supported on a horizontal rotating shaft. The desirable conditions of the rotational motion include the rotational radius (distance from the center of rotation to the center of gravity of the array including the cover member and the substrate) in the range of 12 mm to 150 mm and the rotation speed of not higher than 60 rpm. The rotation speed of not higher than 60 rpm tends to stabilize the shape of the gas accumulation. The rotation speed is preferably not higher than 20 rpm or more preferably not higher than 10 rpm. The rotation speed of not higher than 10 rpm enables stable movement of the gas accumulation in the cavity 12 with little split of the gas shape. More preferably the rotation speed is not higher than 5 rpm. The rotation speed of not higher than 5 rpm ensures stable movement of the gas accumulation in the cavity 12 without even temporary split of the gas shape. The rate of the seesaw motion (one seesaw motion=combination of one upward motion and one downward motion) is preferably 1 to 120 seesaw motions per minute under the condition that the distance from the point of support to the center of gravity of the array including the substrate and the cover member is 0 to 76 mm and the angle of the movement is in the range of 5 degrees to 100 degrees.

Deformation of the elastically deformable opposed area 14 of the cover member 10 by an external force is also effective to move the gas accumulation.

The gas may have any volume that has the sufficient stirring effect. The gas volume is preferably not greater than 50% of the whole volume of the cavity 12. The gas volume of not greater than 50% accelerates the hybridization reaction while limiting the adverse effects of gas bubbles on the hybridization reaction. The gas volume of not greater than 30% is preferable. The gas volume of not greater than 30% accelerates the hybridization reaction while substantially eliminating the adverse effects of gas expansion by heating and expansion of the remaining air on the adhesion surface in the cavity 12. The gas volume of not greater than 15% is more preferable, and even the gas volume of 5% ensures the favorable hybridization reaction.

Another application of the invention is a nucleic acid array that includes the substrate 4 that has a nucleic acid fixation area 6 for fixation of at least one nucleic acid probe, and the cover member 10 that defines a cavity 12, which includes the nucleic acid fixation area 6 of the substrate 4 and has capacity for storage of a liquid for a hybridization reaction of nucleic acid therein. At least part of an area exposed to inside of the cavity 12 forms a hydrophobic region 16. In the nucleic acid array of the invention, the cover member 10 is combined with the substrate 4 to define the cavity 12 for the efficient hybridization reaction. This design ensures the easy and efficient hybridization reaction of nucleic acid. The cover member 10 may be bonded in advance to the substrate 4 or a substrate holder or may be molded integrally with the substrate 4 or the substrate holder, as described previously. The cover member 10 may be integrated with the substrate 4 or the substrate holder in a detachable manner. The nucleic acid array may further have a tab layer 32. The various arrangements, changes, and modifications of the cover member 10 and the substrate 4 described above are applicable to this nucleic acid array.

Still another application of the invention is a hybridization method of nucleic acid. The hybridization method has a hybridization step of implementing the hybridization reaction of an object nucleic acid, which is contained in a hybridization liquid supplied to a cavity 12, with a nucleic acid probe in the cavity 12. The cavity 12 includes a nucleic acid fixation area 6 of the substrate 4 for fixation of the nucleic acid probe and has capacity for storage of the hybridization liquid for the hybridization reaction therein. The hybridization step moves a specific insoluble gas present in the cavity 12 to stir the hybridization liquid. In this hybridization method of the invention, the cavity 12 may not have a hydrophobic region 16. Even without the hydrophobic region 16, the hybridization liquid in the cavity 12 is sufficiently stirred by movement of the insoluble gas. The various motions of the gas or gas accumulation described above are adopted in this hybridization method.

In the hybridization method of the invention, the cavity 12 preferably has concaves and/or convexes in an opposed area 14 facing the nucleic acid fixation area 6 of the substrate 4. In one typical example, the cavity 12 is defined by the substrate 4 and the cover member 10 having concaves and/or convexes on a side of the opposed area 14 facing the nucleic acid fixation area 6 of the substrate 4. The presence of the concaves and/or convexes on the side of the opposed area 14 of the cover member 10 facing the nucleic acid fixation area 6 of the substrate 4 changes the moving range of the gas accumulation in the cavity 12, thus further enhancing the stirring effect.

### EXAMPLES

Some examples of the invention are described below for the better understanding. These examples are only illustrative and are not restrictive in any sense.

### Example 1

In Example 1, enhancement of signal intensity was evaluated for a hybridization device (cover member) having a hydrophobic region. The cover member made of a hydrophobic material was set on a DNA microarray, which was a glass substrate with fixation of cDNAs for hybridization of the cDNAs. The signal intensity of the hybridized product was measured. A cover member of slide glass was used for Comparative Example.

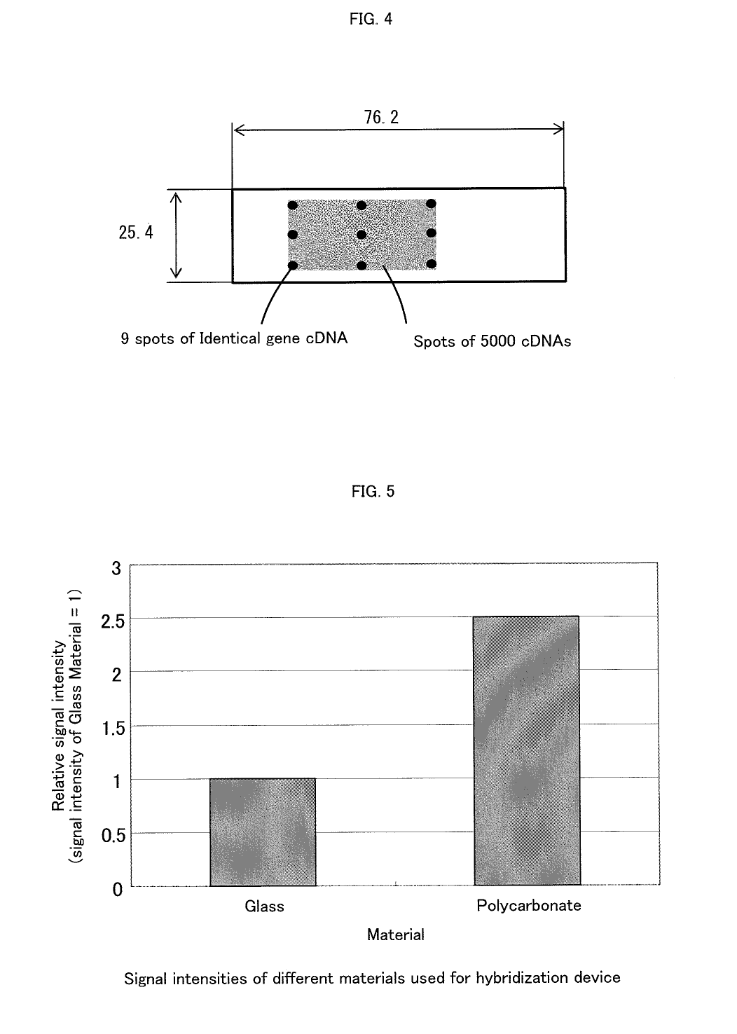

Predetermined amounts of 5000 rat-derived cDNAs prepared in advance were spotted on a poly-L-lysine-coated glass substrate as shown in FIG. 4. One specific gene cDNA among the 5000 cDNAs was spotted at 9 different positions. Each spot diameter of the 5000 spots of the 5000 cDNAs and the 9 spots of the specific gene cDNA was about 150 μm. The glass substrate with the spots of cDNAs was heated at 80° C. for 1 hour, was soaked in a blocking solution (containing 70 mM succinic anhydride, 0.1 M sodium borate (pH 8.0), and 1-methyl-2-pyrrolidone) for 15 minutes. The glass substrate was then soaked in boiled sterilized water for 3 minutes, dehydrated with ethanol, and centrifugally dried to give a DNA microarray.

A Cy3-labeled cDNA was prepared by using 1 μg of rat-derived mRNA for each array according to the procedure written in Saibo Kogaku (Cell Technology) vol. 18, No. 7, p1052-1053 (1999).

As shown in FIG. 2, a 160 μm-thick PET spacer having one adhesive surface and dimensions of 76.2 mm×25.4 mm was placed on one face of a double-sided adhesive film of the identical dimensions. The laminate obtained had an elliptical hole (longer diameter of about 50 mm× shorter diameter of about 20 mm) punched out with an elliptical cutter. A 300 μm-thick polycarbonate film having the dimensions of 76.2 mm×25.4 mm was then attached to the other face of the double-sided adhesive film of the laminate to give a hybridization device of Example 1. The hybridization device attached to the DNA microarray defined an elliptical cavity on the DNA microarray. The hybridization device had two openings for supply of a hybridization liquid at both ends of the elliptical cavity along its longer diameter. A hybridization device of Comparative Example was prepared in the same manner as Example 1 with replacement of the 300 μm-thick polycarbonate film with a 300 μm-thick glass plate. After the hybridization reaction in the hybridization device of Example 1 or in the hybridization device of Comparative Example, the signal intensity was measured by fluorometry and was numerically analyzed.

After attachment of each hybridization device to the DNA microarray, 130 μl of the labeled cDNA (final concentration 5×SSC/0.5% SDS) was injected through one of the two openings, and both the openings were then sealed. The hybridization reaction proceeded while the DNA microarray attached to the hybridization device stood still at 42° C. for 16 hours (without any specific humidity adjustment). After 16 hours, the DNA microarray was detached from the hybridization device, was sequentially washed with a (2×SSC/0.1% SDS) solution, a (1×SSC) solution, and (0.1×SSC) solution by respectively shaking 5 minutes, and was dried by centrifugation (1000 rpm, 3 minutes). The fluorescence was measured with a scanner (Scan Array 4000 manufactured by Packard BioChip Technologies), and the fluorescence intensity was quantified according to a numerical analysis software program (Gene Pix Pro manufactured by Axon). The numerical values of the fluorescence intensities of Example 1 and Comparative Example were compared. The result of the comparison is shown in FIG. 5.

As shown in FIG. 5, the signal intensity by the hybridization device of Example 1 using the polycarbonate film was significantly higher (about 2.5 times) than the signal intensity by the hybridization device of Comparative Example using the slide glass. Namely the hybridization device of Example 1 enhanced the efficiency of the hybridization reaction.

### Example 2

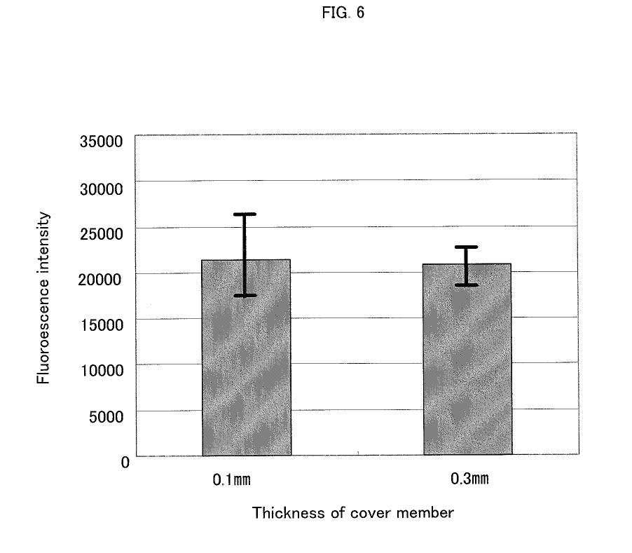

In Example 2, the variation coefficient of signal intensity was evaluated for the different thicknesses in the opposed area of the cover member facing the nucleic acid fixation area of the substrate. The variation coefficient of signal intensity is one effective indication to evaluate the quality of the hybridization. Two hybridization devices were prepared in the same manner as Example 1 by using a 300 μm-thick polycarbonate film and a 100 μm-thick polycarbonate film. The signal intensities were measured at 9 spots of one specific gene cDNA, and the variation coefficient of signal intensity was calculated. The result of the comparison is shown in FIG. 6.

As shown in FIG. 6, the variation coefficient of signal intensity in the hybridization device including the 300 μm-thick opposed area was almost half the variation coefficient of signal intensity in the hybridization device including the 100 μm-thick opposed area. The average signal intensities had no significant difference between these two hybridization devices. This result shows that the sufficiently thick opposed area homogenizes the hybridization reaction over the nucleic acid fixation area and ensures the hybridization reaction of high accuracy and high reproducibility.

### Example 3

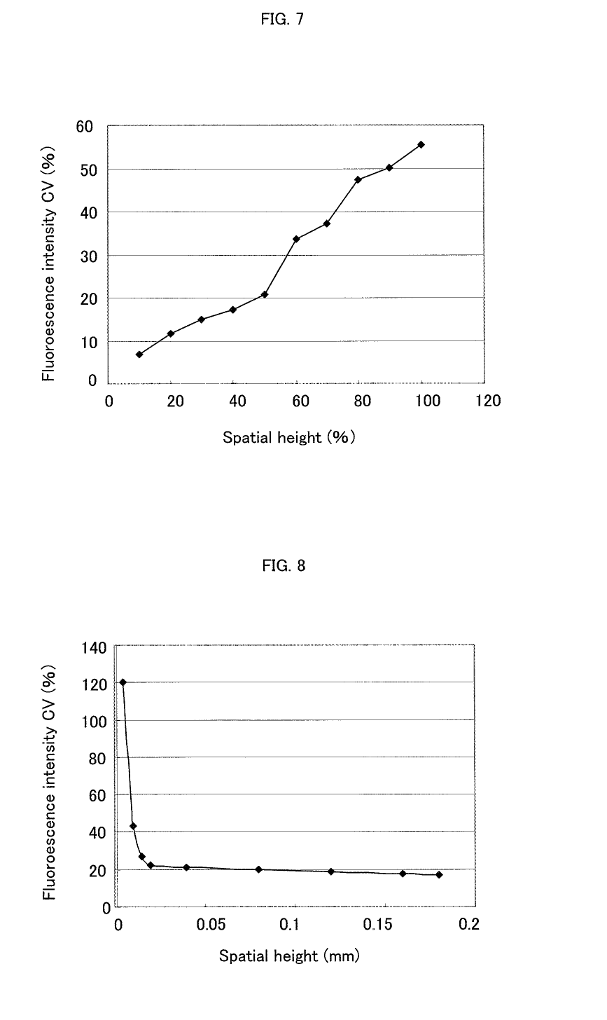

In Example 3, the variation coefficient of signal intensity was evaluated against the variation coefficient of spatial height in the cavity formed by the hybridization device. Hybridization devices prepared as described below were treated in the same manner as Example 1. The signal intensities were measured at 9 spots of one specific gene cDNA, and the variation coefficient of signal intensity was calculated. Ten different hybridization devise were prepared by varying the variation coefficient of spatial height from 10% to 100% at 10% intervals. Each hybridization device had a laminate of 160 μm-thick spacer and a polycarbonate film having an average film thickness of 300 μm. An area of about 30 mm×10 mm in the polycarbonate film corresponding to the opposed area was deformed to be projected toward the DNA microarray. The variation coefficient of spatial height was calculated from the maximum spatial height and the minimum spatial height of the cavity. The average spatial height and the variation coefficient of spatial height were determined by the height-surface undulation measurement described above. The cavity was divided into eight equal parts by 7 parting lines in the vertical direction and into four equal parts by 3 parting lines. The peripheral heights and the maximum and minimum values of the surface undulation were measured on each of the parting lines. The peripheral heights were measured with a digital micrometer (Digimicro manufactured by Nikon Corporation), and the surface undulations were measured with a surface texture and contour measuring instrument (Surfcom manufactured by Tokyo Seimitsu Co., Ltd). The result of the measurement is shown in FIG. 7.

As the general tendency, the variation coefficient of signal intensity increased with an increase in variation coefficient of spatial height as shown in FIG. 7. The variation coefficient of signal intensity had a smaller rate of increase and was restricted to or below 20% in the range of the variation coefficient of spatial height of not greater than 50%. The variation coefficient of signal intensity had a greater rate of increase and significantly increased far beyond 20% in the range of the variation coefficient of spatial height of greater than 50%. This result shows that the favorable variation coefficient of spatial height in the cavity is not greater than 50%.

### Example 4

In Example 4, the variation coefficient of signal intensity was evaluated against the spatial height (dimension) in the cavity formed by the hybridization device. Hybridization devices prepared as described below were treated in the same manner as Example 1. The signal intensities were measured at 9 spots of one specific gene cDNA, and the variation coefficient of signal intensity was calculated. Nine hybridization devices were prepared to have the variation coefficient of spatial height equal to 50%. The hybridization devices respectively had spacers of different thicknesses 5 μm, 10 μm, 15 μm, 20 μm, 40 μm, 80 μm, 120 μm, 160 μm, and 180 μm placed on a polycarbonate film having an average film thickness of 300 μm. An area of about 30 mm×10 mm in the polycarbonate film corresponding to the opposed area was deformed to be projected toward the DNA microarray. The variation coefficient of spatial height was calculated from the maximum spatial height and the minimum spatial height of the cavity. The average spatial height and the variation coefficient of spatial height were determined by the same procedure as Example 3. The result of the measurement is shown in FIG. 8.

As shown in FIG. 8, the variation coefficient of signal intensity drastically increased with a decrease in spatial height of the cavity. The variation coefficient of signal intensity was not greater than 30% in the range of the spatial height of not less than 15 μm and was almost constant to 20% in the range of the spatial height of not less than 20 μm. This result shows that the spatial height of the cavity in the range of 15 μm to 200 μm well reduces the variation coefficient of signal intensity and thus ensures the hybridization reaction of high reproducibility.

### Example 5

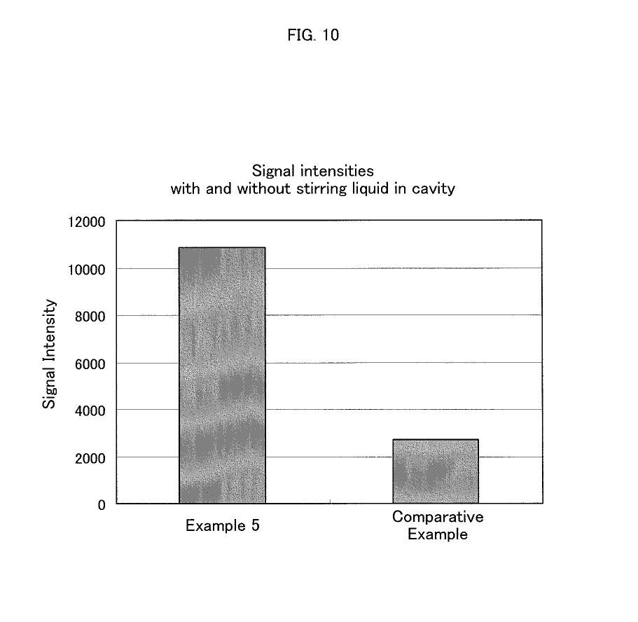

In Example 5, the signal intensity was evaluated with or without stirring (rotational motion) the liquid in the cavity formed by the hybridization device. The hybridization device of Example 5 was prepared and treated in the same manner as Example 1, except the hybridization conditions given below. The fluorescence intensity was quantified as described above. The injected amount of the labeled cDNA was 110 μl. At the hybridization temperature of 60° C., the DNA microarray (substrate) and the hybridization device were set in a rotating hybridization machine (hybridization incubator HB-100 manufactured by Taitec Corporation) for the hybridization time of 16 hours. The center of gravity of the microarray was placed at a radial distance of approximately 75 mm from a rotating shaft of a rotary unit in the hybridization incubator. The rotation speed was 4 rpm. Under these rotational conditions, the air accumulation (about 20 μl, 15% by volume) substantially kept its shape and slowly moved in the cavity of the microarray during the hybridization reaction. As Comparative Example, the microarray was subjected to the similar hybridization conditions without the rotational motion and stood still for the hybridization time. The fluorescence intensity was then quantified by the above procedure. The result of the comparison is shown in FIG. 10.

As shown in FIG. 10, the signal intensity of Example 5 with the rotational motion of the hybridization device and the substrate was approximately 5 times as high as the signal intensity of Comparative Example. The variation coefficient of signal intensity was 5% in Example 5 and 13% in Comparative Example. This results show that the rotational motion of the substrate and the hybridization device to move the air (gas) accumulation in the cavity defined by the substrate and the hybridization device effectively increases the hybridization efficiency and enhances the signal intensity. The enhanced signal intensity makes significant contribution to the high accuracy and the high reproducibility. This stirring motion thus enhances the accuracy and the reproducibility.

The hybridization operations of Example 5 and Comparative Example were performed with various injection amounts of the labeled cDNA, 65 μl, 90 μl, and 113.5 μl to vary the air volume in the cavity to 5% by volume, 30% by volume, and 50% by volume. The fluorescence intensities were then quantified by the above procedure. In any of these air volume conditions, the signal intensity of Example 5 was approximately 5 times as high as the signal intensity of Comparative Example. This result suggests the high hybridization efficiency in the presence of the air accumulation in the range of 5% by volume to 50% by volume.

### Example 6

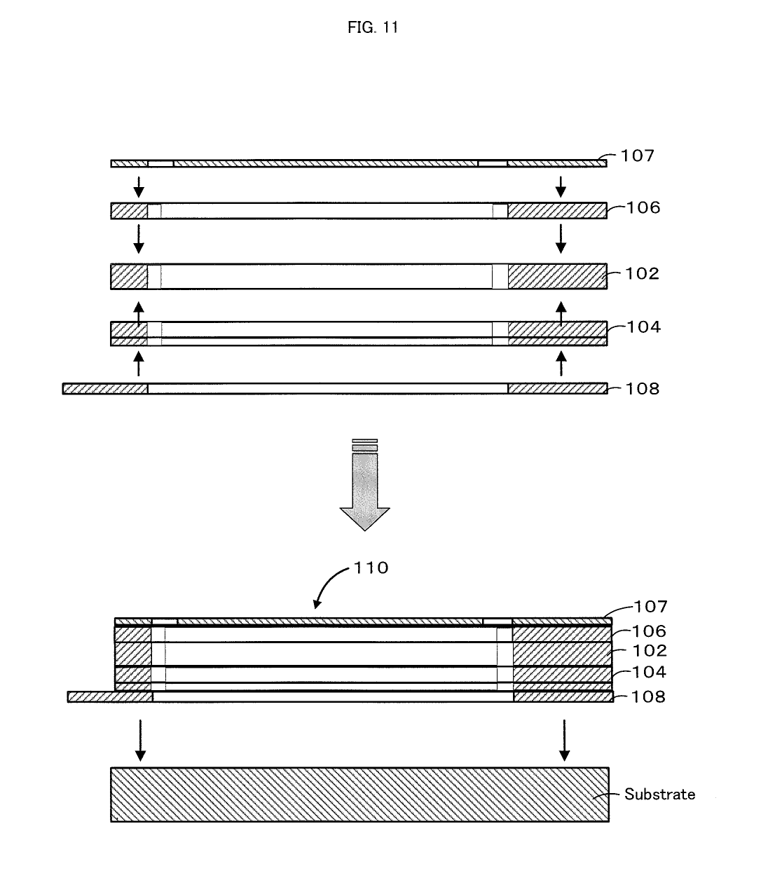

In Example 6, the signal intensity was evaluated with or without stirring (seesaw motion) the liquid in the cavity formed by the hybridization device. The hybridization device of Example 6 used a cover member 110 of the structure shown in FIG. 11 to define a cavity having a volume of 400 μl. The hybridization device of Example 6 was treated in the same manner as Example 1, except the hybridization conditions given below. The fluorescence intensity was quantified as described above.

The manufacturing process of the hybridization device (cover member 110) of Example 6 first cut out an acrylic resin plate to an acrylic resin spacer 102 and made sealing members 104 and 106 punched out to the same shape as the acrylic resin spacer 102. The sealing member 104 had a single-sided sealing element having a detachable adhesive layer and a double-sided sealing element integrated with the single-sided sealing element. The sealing member 106 had only a double-sided sealing element. The manufacturing process then bonded the sealing member 104 to one face of the acrylic resin spacer 102 and the sealing member 106 to the other face of the acrylic resin spacer 102 to prepare a laminate body. A 0.3 mm-thick polycarbonate film 107 with openings perforated in advance was further laid on the sealing member 106 of the laminate body. A silicone rubber element 108 having a contour slightly extended from the contour of the acrylic resin spacer 102 was prepared and was bonded to the detachable adhesive layer of the sealing member 104 to have a fringe around the periphery of the acrylic resin spacer 102. The resulting cover member 110 had the total thickness of 5 mm. The cover member 110 was attached to a microarray (substrate) prepared by the procedure of Example 1 and was left in vacuum (−98 kPa) for at least 30 minutes. This completed a reaction cavity defined by the hybridization device of Example 6 and the substrate.

The injected amount of the labeled cDNA was 200 μl. At the hybridization temperature of 60° C., the DNA microarray (substrate) and the hybridization device were set in a seesaw hybridization machine (benchtop rocker (35/35D) manufactured by Labnet International Inc.) for the hybridization time of 16 hours. The microarray (substrate) and the hybridization device were seesawed in an angle range of ±20 degrees and at a seesaw motion rate of 50 sets/minute. The microarray was placed to set its center of gravity at the point of support of the seesaw motion. Under these seesaw conditions, the air accumulation (about 200 μl, 50% by volume) substantially kept its shape and slowly moved in the cavity of the microarray during the hybridization reaction. As Comparative Example, the microarray was subjected to the similar hybridization conditions without the seesaw motion and stood still for the hybridization time. The fluorescence intensity was then quantified by the above procedure.

The signal intensity of Example 6 with the seesaw motion of the hybridization device and the substrate was approximately 5 times as high as the signal intensity of Comparative Example. This result shows that the seesaw motion of the substrate and the hybridization device to move the air (gas) accumulation in the cavity defined by the substrate and the hybridization device effectively increases the hybridization efficiency and enhances the signal intensity. The enhanced signal intensity makes significant contribution to the high accuracy and the high reproducibility. This stirring motion thus enhances the accuracy and the reproducibility.

The cover member 110 of Example 6 had the silicone rubber element 108 functioning as a tab layer. This structure enabled the cover member 110 to be easily detached from the substrate with a small force, that is, without much loading.

The present application claims priority from Japanese patent application No. 2004-221807 filed on Jul. 29, 2004, the contents of which are hereby incorporated by reference into this application.

1. A hybridization device for a hybridization reaction of nucleic acid, said hybridization device comprising:
a cover member that defines a cavity, which includes a nucleic acid probe fixation area of a substrate for fixation of a nucleic acid probe and has capacity for storage of a liquid for the hybridization reaction therein,
wherein at least part of an area exposed to inside of the cavity forms a hydrophobic region.

2. A hybridization device according to claim 1, wherein the hydrophobic region is formed in at least part of the cover member.

3. A hybridization device according to claim 1, wherein the cover member has the hydrophobic region in an area opposed to the nucleic acid probe fixation area.

4. A hybridization device according to claim 1, wherein the hydrophobic region has a water contact angle of not less than 30 degrees.

5. A hybridization device for a hybridization reaction of nucleic acid, said hybridization device comprising:
a cover member that defines a cavity, which includes a nucleic acid probe fixation area of a substrate for fixation of a nucleic acid probe and has capacity for storage of a liquid for the hybridization reaction therein,
wherein an opposed area of the cover member facing the nucleic acid probe fixation area has a thickness of not less than 300 μm.

6. A hybridization device according to claim 5, wherein the cover member has a hydrophobic region formed in at least part of an area exposed to inside of the cavity.

7. A hybridization device for a hybridization reaction of nucleic acid, said hybridization device comprising:
a cover member that defines a cavity, which includes a nucleic acid probe fixation area of a substrate for fixation of a nucleic acid probe and has capacity for storage of a liquid for the hybridization reaction therein,
wherein the nucleic acid probe fixation area included in the cavity has a variation coefficient of spatial height of not higher than 50%.

8. A hybridization device according to claim 7, wherein an average spatial height is not less than 15 μm.

9. A hybridization device according to claim 7, wherein the cover member has a hydrophobic region formed in at least part of an area exposed to inside of the cavity.

10. A hybridization device according to claim 1, wherein the cover member has at least a sheet element and a spacer to be interposed between the sheet element and the substrate.

11. A hybridization device according to claim 1, wherein the cover member has an opening for supply of the liquid into the cavity in an area opposed to the nucleic acid probe fixation area, and
the opening is configured to have a projected portion formed by outward extension of an inner circumferential wall of the cavity.

12. A hybridization device according to claim 11, wherein the cover member has multiple openings located on both ends of the cavity in a longitudinal direction.

13. A hybridization device according to claim 1, wherein an opposed area of the cover member facing the nucleic acid probe fixation area is composed of one or multiple materials selected from the group consisting of polycarbonates, polyolefins, polyamides, polyimides, acrylic resins, fluorides thereof, and poly(vinyl halides).

14. A hybridization device according to claim 1, wherein an opposed area of the cover member facing the nucleic acid probe fixation area has at least either of a concavity and a convexity.

15. A hybridization device according to claim 1, wherein the cover member is integrated with the substrate in a detachable manner and has a tab layer with a holdable, extended end from the substrate and the cover member.

16. A hybridization method of nucleic acid, said hybridization method comprising:
a setting step of setting a hybridization device for a hybridization reaction of nucleic acid according to claim 1 to a substrate with a nucleic acid probe fixation area for fixation of a nucleic acid probe; and
a hybridization step of implementing a hybridization reaction of an object nucleic acid, which is contained in a liquid supplied to a cavity including the nucleic acid probe fixation area, with the nucleic acid probe in the cavity.

17. A hybridization method according to claim 16, wherein the hybridization step implements the hybridization reaction while the substrate and the hybridization device stand still.

18. A hybridization method according to claim 17, wherein the hybridization step stirs the liquid in the cavity.

19. A hybridization method according to claim 18, wherein the hybridization step implements the hybridization reaction in the presence of a specific gas in the cavity.

20. A hybridization method according to claim 19, wherein the hybridization step moves the specific gas in the cavity to stir the liquid.

21. A hybridization method according to claim 18, wherein the hybridization step implements the hybridization reaction while the substrate and the hybridization device defining the cavity is moved.

22. A hybridization method of nucleic acid, said hybridization method comprising:
a hybridization step of implementing a hybridization reaction of an object nucleic acid, which is contained in a liquid supplied to a cavity, with a nucleic acid probe in the cavity, where the cavity includes a nucleic acid probe fixation area of a substrate for fixation of the nucleic acid probe and has capacity for storage of the liquid for the hybridization reaction therein,
wherein the hybridization step moves a specific gas present in the cavity to stir the liquid.

23. A hybridization reaction kit for a hybridization reaction of nucleic acid, said hybridization reaction kit comprising:
a substrate that has a nucleic acid probe fixation area for fixation of a nucleic acid probe; and
a cover member that defines a cavity, which includes the nucleic acid probe fixation area of the substrate and has capacity for storage of a liquid for the hybridization reaction therein,
wherein at least part of an area exposed to inside of the cavity forms a hydrophobic region.

24. A hybridization reaction kit according to claim 23, wherein the cover member is integrated with the substrate in a detachable manner and has a tab layer with a holdable, extended end from the substrate and the cover member.

25. A nucleic acid array, comprising:
a substrate that has a nucleic acid probe fixation area with at least one nucleic acid probe fixed therein; and
a cover member that defines a cavity, which includes the nucleic acid probe fixation area of the substrate and has capacity for storage of a liquid for a hybridization reaction of nucleic acid therein,
wherein at least part of an area exposed to inside of the cavity forms a hydrophobic region.

26. A nucleic acid array according to claim 25, wherein the cover member is detachably attached to the substrate.

27. A nucleic acid array according to claim 25, wherein the cover member is integrated with the substrate in a detachable manner, and
the cover member and the substrate are laid one upon the other via a tab layer having a holdable, extended end from the substrate and the cover member.
